(19) United States
(12) Patent Application Publication
(10) Pub. No.: US 2005/0080380 A1
(43) Pub. Date: Apr. 14, 2005

(54) Safety syringe

(76) Inventors: Hsin-Po Hsieh, Chian-Hwa Hsien (TW); Chi-Zer Ho, Taipei (TW); Shih-Chun Wang, Chia-Yi (TW)

Correspondence Address: Rosenberg, Klein & Lee;Suite 101, 3458 Ellicott Center Drive, Ellicott City, MD, 21043, US

(21) Appl. No.: 10/683,401

(22) Filed: Oct. 14, 2003

(51) Int. Cl. A61M 5/32

(52) U.S. Cl. 604/197; 604/110

(57) ABSTRACT

A safety syringe has a hollow barrel, a seal, a needle hub and a plunger. The plunger is mounted in the hollow barrel and has a needle retractor at one end. The seal has an axial circular through hole that is mounted around the plunger and has multiple detents. The hollow barrel has an inside surface and the seal has an outside surface, which correspond to each other and are non-circular. The plunger has detent protrusions corresponding to the detents in the seal to selectively hold the plunger is position. The needle hub with a needle is detachably mounted inside the hollow barrel and has an axial locking opening to engage the plunger. Since the plunger will not accidentally rotate, the protruding head on the plunger can be inserted precisely into the locking chamber of the needle hub and either engage or be withdrawn from the axial locking opening.

### BACKGROUND OF THE INVENTION

1. Field of the Invention

The present invention relates to a syringe, and more particularly to a safety syringe that can safely hold a used needle and prevent the syringe from being used more than once.

2. Description of Related Art

To keep doctors, nurses or workers who deal with discarded syringes from being injured or infected by used needles, safety syringes with retractable needles have been designed. A conventional safety syringe has a hollow barrel, a plunger and a needle hub with a needle. The needle hub of the conventional safety syringe has an axial locking chamber, and the plunger has a proximal end and a protruding head. The protruding head is formed on the proximal end of the plunger. When injecting medicine into a body, the proximal end of the plunger is pushed toward the needle hub, and the protruding head of the plunger is engages the axial locking chamber in the needle hub. When a user pulls the plunger back, the needle hub will be pulled inside the hollow barrel to protect people from being injured by the needle.

However, before the conventional safety syringe is used, the needle hub is often pulled inside the hollow barrel. Because the user must push the plunger totally inside the hollow barrel to exhaust air, the protrusion head easily engages the axial locking chamber making the safety syringe useless.

With reference to FIG. 6, a conventional safety syringe has a hollow barrel (50), a needle hub (61) with a needle (60) and a plunger (70). The plunger (70) has a proximal end (not numbered), a seal (not numbered) and a protruding head (71) formed on the proximal end. The needle hub (61) has an axial locking chamber (62) that has a shape the same as the shape of the protruding head (71) and corresponds to the protruding head (71). To overcome the previously identified problems with the conventional safety syringes, the plunger (70) is initially rotated so the protruding head (71) is not aligned with and will not engage the axial locking chamber (62). After the safety syringe is used, the plunger (70) is rotated to align and engage the protruding head (71) with the axial locking chamber (62). Then the plunger (70) is pulled into the hollow barrel (50) with the needle hub (61).

Because the hollow barrel (50), proximal end of the plunger (70) and the seal are cylindrical, the plunger (70) and the seal are easily rotated when the plunger (70) is inserted into the hollow barrel (50) so the protruding head (71) inadvertently and accidentally aligns with the axial locking chamber (62). Furthermore, the precise shape of the protruding head (71) and the axial locking chamber (62) may make inserting the protruding head (71) into the axial locking chamber (62) difficult. Specifically, the plunger (70) does not have any feature to align the protruding head (71) with the axial locking chamber (62), and the needle hub (61) easily separates from the plunger (70).

To overcome the shortcomings of conventional safety syringes, the present invention provides a safety syringe to mitigate or obviate the aforementioned problem.

### SUMMARY OF THE INVENTION

The primary objective of the present invention is to provide a syringe that is safer and more convenient to use and handle than a conventional safety syringe. The safety syringe in accordance with the present invention has a hollow barrel, a seal, a needle hub, a plunger and features incorporated into these main elements to make the syringe safer and more convenient to use and handle than conventional safety syringes. The plunger is mounted in the hollow barrel and has a proximal end and an asymmetrical needle retractor formed on the proximal end. The hollow barrel has a non-circular interior surface, and the seal has a corresponding non-circular outside surface and a circular interior surface corresponding to a circular surface around the proximal end of the plunger. The seal is mounted around the proximal end of the plunger, and corresponding detents and protrusions on the interior surface of the seal and the outside surface of the proximal end of the plunger hold the plunger and needle retractor in position. The needle hub has a needle, is detachably mounted inside the proximal end of the hollow barrel and has an axial fitting to connect to the needle retractor on the plunger.

When the safety syringe is used, the shape of the seal mounted in the plunger interior surface keeps the plunger from rotating so the needle retractor on the plunger will not inadvertently or accidentally engage the axial fitting. Furthermore, the plunger can be rotated intentionally inside the seal so the needle retractor precisely aligns with and engages the axial fitting. When the plunger with the needle has been pulled into the hollow barrel, notches in the plunger allow the plunger to be broken off so the needle cannot be inadvertently pushed back out of the hollow barrel. With the needle completely and securely inside the hollow barrel, people cannot be injured by the needle.

Other objectives, advantages and novel features of the invention will become more apparent from the following detailed description when taken in conjunction with the accompanying drawings.

### BRIEF DESCRIPTION OF THE DRAWINGS

FIG. 1 is a partial perspective view of a safety syringe in accordance with the present invention;

FIG. 2 is a cross-sectional front plan view of the safety syringe in FIG. 1 through the seal;

FIG. 3 is an enlarged perspective view of the safety syringe in FIG. 1;

FIG. 4 is an enlarged perspective view of the plunger attached to the needle hub in FIG. 1;

FIG. 5 is an operational perspective view of the safety syringe in FIG. 1 with the needle retractor on the plunger connected to the axial fitting in the needle hub; and

FIG. 6 is a perspective view of a conventional safety syringe in accordance with prior art.

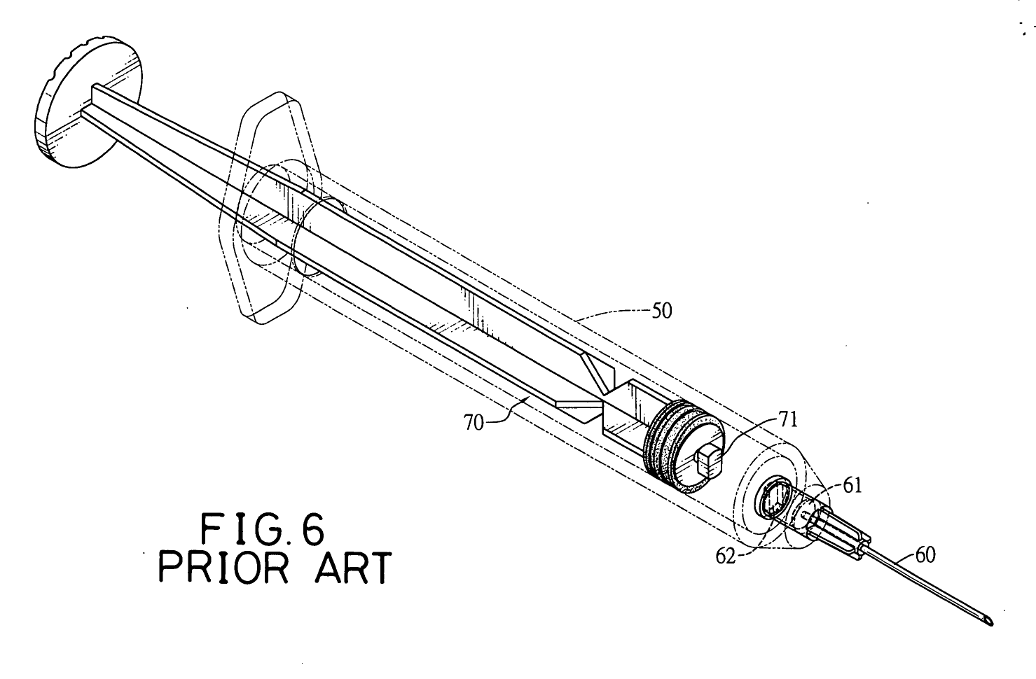

### DETAILED DESCRIPTION OF THE PREFERRED EMBODIMENT

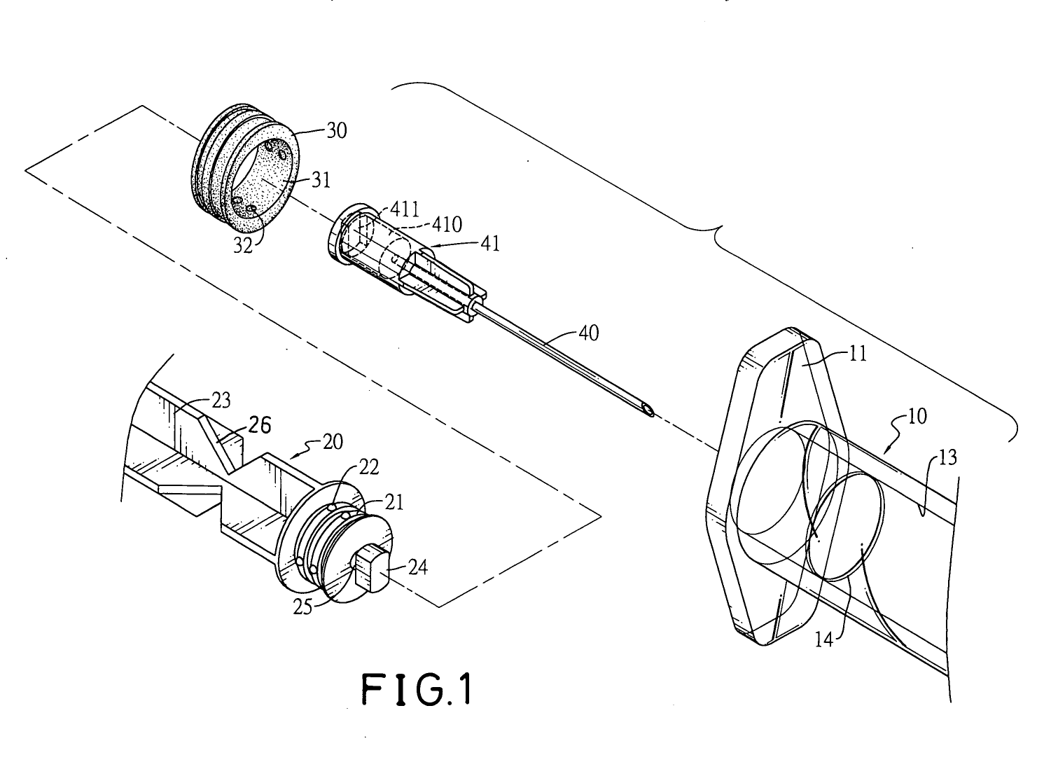

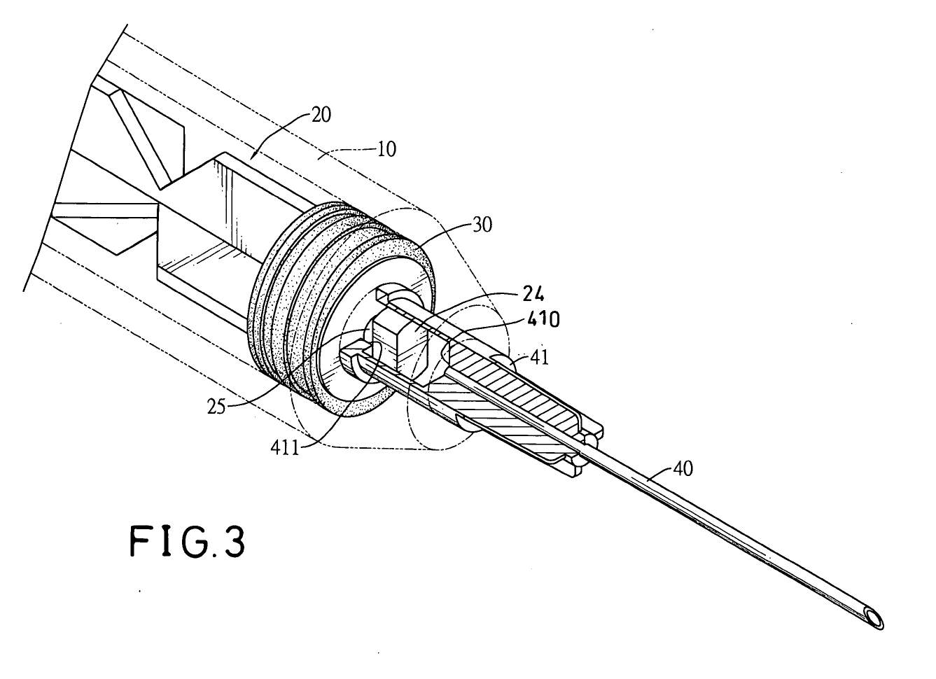

With reference to FIGS. 1 and 3, a safety syringe in accordance with the present invention has a hollow barrel (10), a plunger (20), a seal (30) and a needle hub (41) with a needle (40).

The hollow barrel (10) is cylindrical and has a distal end (not numbered), a proximal end (not numbered), a flange (11), a non-circular interior surface (13), such as an oval cylinder, a conical end (not numbered) and an annular recess (14). The flange (11) is defined radially around and extends out from the distal end of the hollow barrel (10). The conical end has an opening (not numbered) and is formed at the proximal end of the hollow barrel (10). The annular recess (14) is defined on the interior surface (13) of the hollow barrel (10) near the distal end.

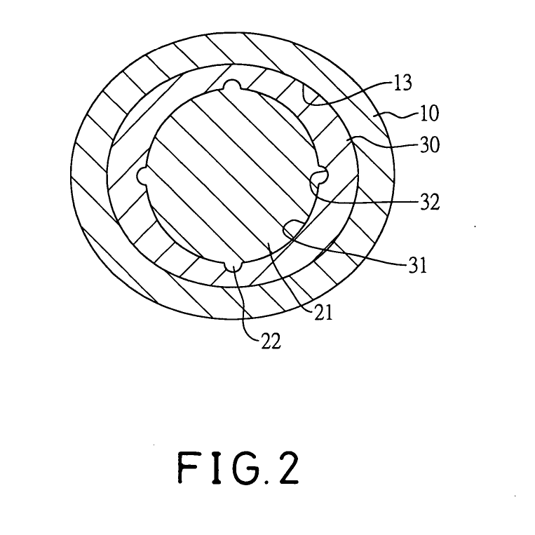

With further reference to FIG. 2, the seal (30) is made of resilient material and has a noncircular outside surface (not numbered), an axial through hole (not numbered), an inner surface (31) and multiple detents (32). The non-circular interior surface (13) of the hollow barrel (10) is smaller than the outside surface of the seal (30). The non-circular interior surface (13) and the outside surface of the seal (30) are similar so the seal (30) can align with and be slidably mounted inside the hollow barrel (10) and will not rotate inside the hollow barrel (10). The outer surface of the seal (30) has an annular lip (not numbered) formed on the outer surface to engage the annular recess (14) in the hollow barrel (10). The multiple detents (32) are formed on the inner surface (31) and are evenly spaced. As an example, adjacent detents (32) in the embodiment described are separated by an angle of 90° with each other.

The plunger (20) has a proximal end (not numbered), a front head (21), multiple axial ribs (23), multiple notches (26) and a needle retractor (not numbered). The front head (21) is integrally formed on the proximal end of the plunger (20) and has an end surface (not numbered), an annular surface (not numbered) and multiple detent protrusions (22) that are formed to correspond to the detents (32) inside the seal (30). The needle retractor may consist of an axial protruding lock (24) and a neck (25) formed between the axial protruding lock (24) and the end surface of the front head (21). The axial protruding lock (24) is not spherical. The seal (30) is mounted around annular surface on the front head (21), and the axial protruding lock (24) extends out of the through hole in the seal (30). A notch (26) is defined on each axial rib (23) near the proximal end.

The needle hub (41) with the needle (40) is mounted detachably inside the conical end of the hollow barrel (10) so the needle (40) extends out of the opening. The needle hub (41) has a distal end (not numbered), an annular lip (not numbered), a locking opening (411), an outside surface (not numbered) and an axial locking chamber (410). The annular lip is formed on the distal end of the needle hub (41) and the locking opening (411) is defined on the annular lip to engage with the protruding head (24). The axial passage is defined through the needle hub (41), and one end of the needle (40) is inserted into the axial passage of the needle hub (41). The needle (40) has a fluid passage (not numbered) defined axially through the needle (40). The axial locking chamber (410) is defined inside the needle hub (40), and the axial passage communicates with the axial locking chamber (410) and the inner space in the hollow barrel (10).

The seal (30) is mounted on the front end (21) of the plunger (20) and is held in place by the detent protrusions (22) inside the detents (32). When the plunger (20) with the seal (30) is inserted inside the hollow barrel (10), the protruding head (24) can be inserted into the locking opening (411) without engaging the locking opening (411) to draw medicine or blood into the hollow barrel (10).

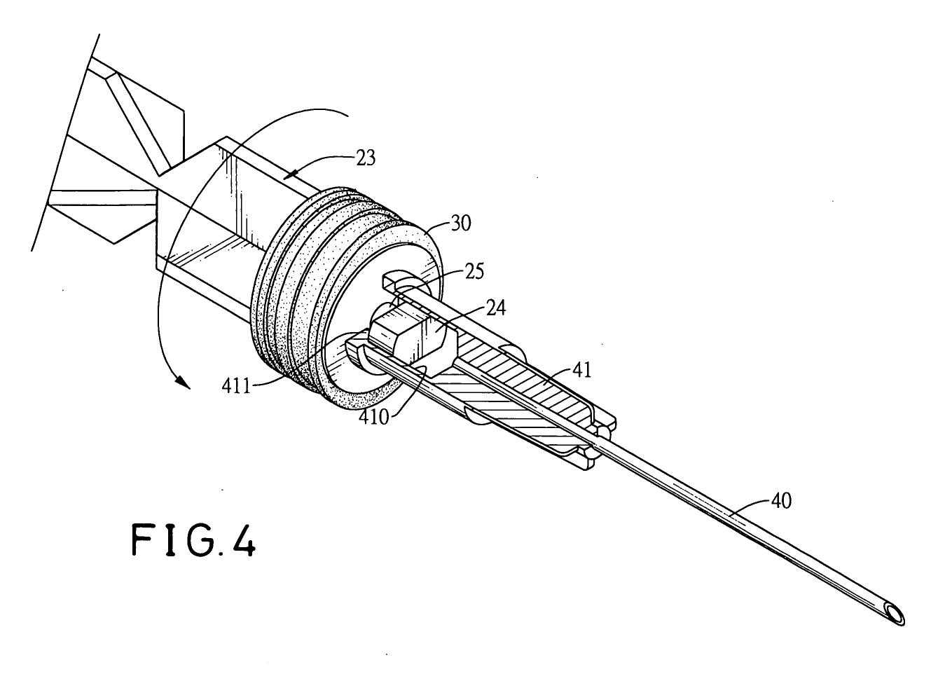

With further reference to FIG. 4, the protruding head (24) on the plunger (20) is pressed into the locking opening (411) in the needle hub (41) and rotated to lock the plunger (20) to the needle hub (41). Because the outside surface of the seal (30) and the non-circular interior surface (13) of the hollow barrel (10) are similar, rotating the plunger (20) will not allow the seal (30) to rotate with the plunger (20), and the detent protrusions (22) on the plunger (20) will move to adjacent detents (32) in the seal (30).

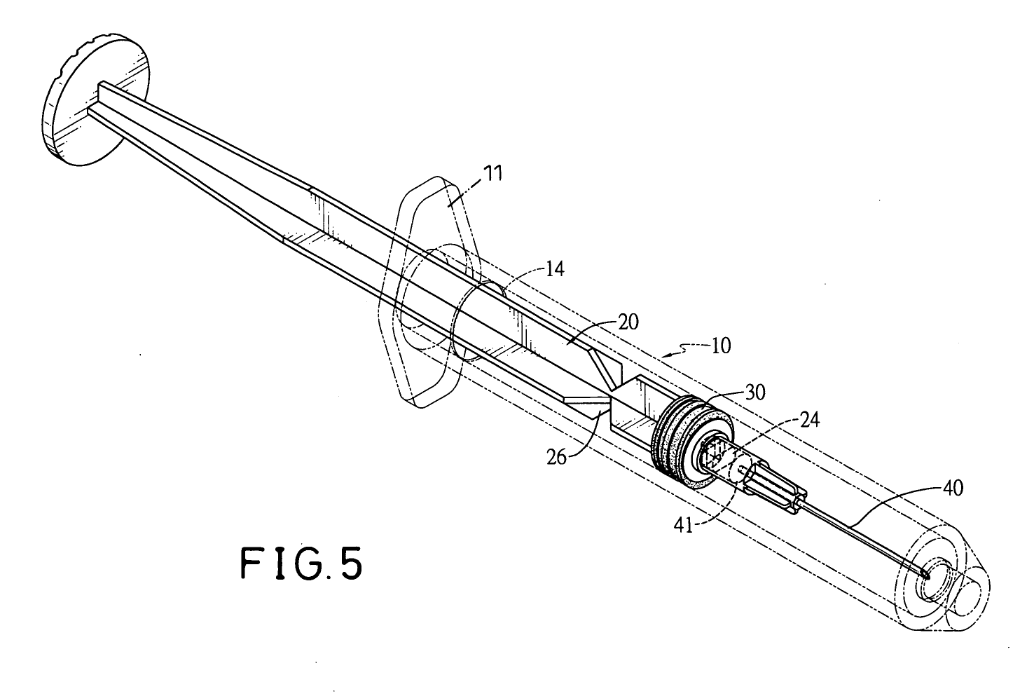

With reference to FIG. 5, the protruding head (24) engages the locking opening (411), and retracting the plunger (20) pulls the needle hub (41) and the needle (40) inside the hollow barrel (10). When the annular lip on the seal (30) moves inside the annular recess (14) in the hollow barrel (10), the notches (26) in the plunger (20) are aligned with the flange (11). The plunger (20) is broken at the notches (26) to prevent the safety syringe from being used again.

The safety syringe in accordance with the present invention has the following advantages.

1. The outside surface of the seal (30) mounted in the plunger (20) is non-circular and corresponds to the non-circular interior surface (13) of the hollow barrel (10) so the seal (30) will not rotate. The protruding head (24) on the plunger (20) can be inserted precisely into the locking opening (411) in needle hub (41).

2. The plunger (20) can be broken at the notches (26) of the axial ribs (23) so the syringe cannot be used again.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

1. A safety syringe comprising a hollow barrel, a plunger, a seal and a needle hub;
the hollow barrel being cylindrical and having
a distal end;
a proximal end;
a flange defined radially around and extending out from the distal end;
a non-circular interior surface;
an annular recess defined inside the hollow barrel near the distal end; and
a conical end integrally formed with the proximal end of the hollow barrel and having
an opening define through the conical end;
the seal mounted slidably inside the hollow barrel and having
a through hole;
an outer surface that is non-circular and corresponds to the interior surface of the hollow barrel;
a circular inner surface;
multiple detents defined on the inner surface; and
an annular lip formed on the outer surface to be selectively mounted inside the annular recess in the hollow barrel;
the plunger connected to the seal and having
a proximal end;
a front head integrally formed on the proximal end of the plunger with a top surface having multiple detent protrusions corresponding to the detents in the seal;
a needle retractor formed on the top surface of the front head;
multiple axial ribs; and
multiple notches defined respectively on the axial ribs; and
the needle hub having a needle and being detachably mounted inside the proximal open end of the hollow barrel and having
a distal end;
an annular lip formed on the distal end;
a locking opening defined in the annular lip;
an axial locking chamber; and
a fluid passage communicating with the axial locking chamber in the hollow barrel;
wherein the non-circular outside surface of the seal corresponds to the non-circular interior surface of the hollow barrel and is aligned with the hollow barrel.

2. The safety syringe as claimed in claim 1, wherein the needle retractor of the plunger is an axial protruding lock and the shape of the locking opening corresponds to the axial protruding lock to receive and engage the axial protruding lock.

3. The safety syringe as claimed in claim 1, wherein adjacent detents defined in the seal are 90° apart.
